(19) United States
(12) Plant Patent Application Publication
(10) Pub. No.: US 2007/0079408 P1
(43) Pub. Date: Apr. 5, 2007

(54) Loblolly pine tree named 'CF Q3802'

(76) Inventors: John Pait, Atlanta, GA (US); Plamen Denchev, Victoria (CA); Stephen Attree, Victoria (CA); Margarita Gilbert, Sidney (CA)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 11/245,267

(22) Filed: Oct. 5, 2005

(51) Int. Cl. A01H 7/00

(52) U.S. Cl. PLT/213

(57) ABSTRACT

A new and distinctive variety of a loblolly pine tree which has been denominated varietally as ‘CF Q3802’ which is distinguished by distinctive genetic fingerprint, high resistance to fusiform rust, outstanding stem straightness, long internodes and distinctive narrow crown with slender branches, and extremely fast growth.

### LATIN NAME

Pinus taeda

### VARIETY DENOMINATION

CF Q3802

### BACKGROUND

A new variety of loblolly pine tree (Pinus taeda), has been discovered. This selection has been designated as CF Q3802.

This new variety is a progeny of a second generation selection pollinated by a first generation selection. Female parent is an open pollinated progeny of Georgetown County, South Carolina first generation selection. Male parent is a first generation selection made in Barnwell County, South Carolina.

Cross pollination occurred in early 1997 followed by induction and cryopreservation of embryogenic tissue in 1998. First somatic seedlings were produced in 1999 and planted in early 2000 in seven field experiments. A total of 56 ramets were planted ranging from five to eleven ramets per field experiment. Additional ramets were produced and planted in 2001 (22 ramets in six field experiments) and in 2002 (48 ramets in six field experiments). The field experiments are located in Mississippi, Florida, Georgia and South Carolina.

### BRIEF SUMMARY

A new and distinct cultivar of loblolly pine (Pinus taeda) is distinctly characterized by great resistance to fusiform rust, superior growth and outstanding stem straightness, long internodes and distinctive narrow crown with slender branches, and which is mature for commercial harvesting sooner than conventionally grown trees under the ecological conditions prevailing in the Piedmont, Atlantic and Gulf Coastal Plains, and Mid-Continent regions of the United States.

The Pinus taeda plants of this variety were asexually propagated using an advanced form of micropopagation called somatic embryogenesis carried out at CellFor's production facility in Victoria, Canada. Somatic embryogenesis uses a complex process which relies on the splitting of one embryo into many identical embryos. Somatic embryos can then be grown into plants which are all identical genetically. The asexual propagation occurs at an earlier stage in the plant's life cycle than most other micropropagated plants. The detailed methods for somatic embryogenesis used for asexually propagating conifers in general are described in U.S. Pat. No. 6,372,496 and for loblolly pine in particular in US Patent Application 2004/0203150.

### BRIEF DESCRIPTION OF THE DRAWINGS

FIG. 1 is a photograph showing CF Q3802 (to the right), at age 5 in Brewer, Miss., with a low number of whorls, narrow crown, and stem straightness, when compared to the tree to the left.

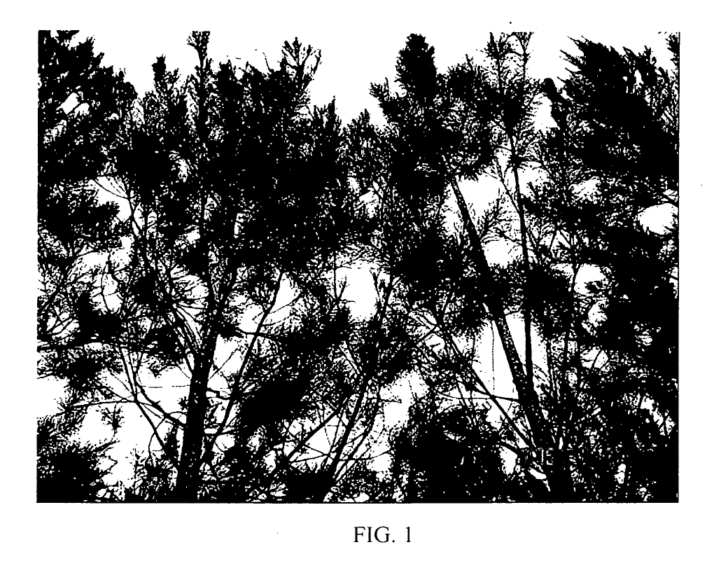

FIG. 2 is a photograph showing CF Q3802, at age 5 in Brewer, Miss., with low number of whorls and slender branches.

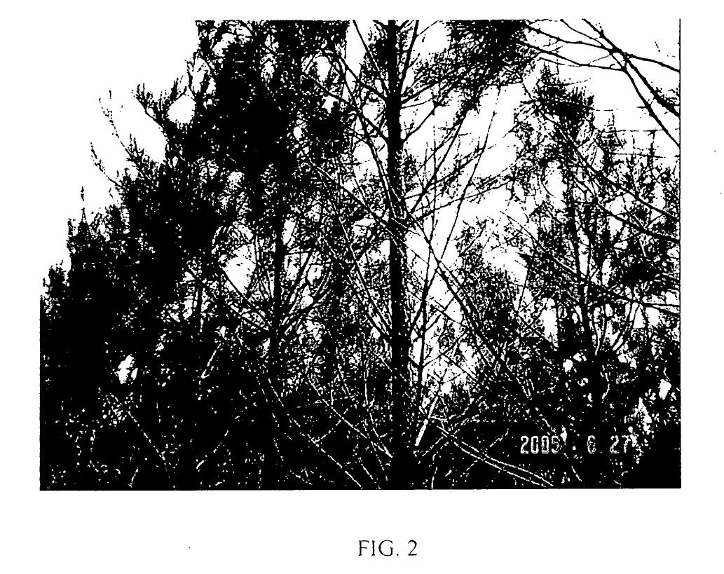

FIG. 3 is a photograph showing CF Q3802, at age 3 in Shubuta, Miss., with low number of whorls, stem straightness, and average to steep branch angle.

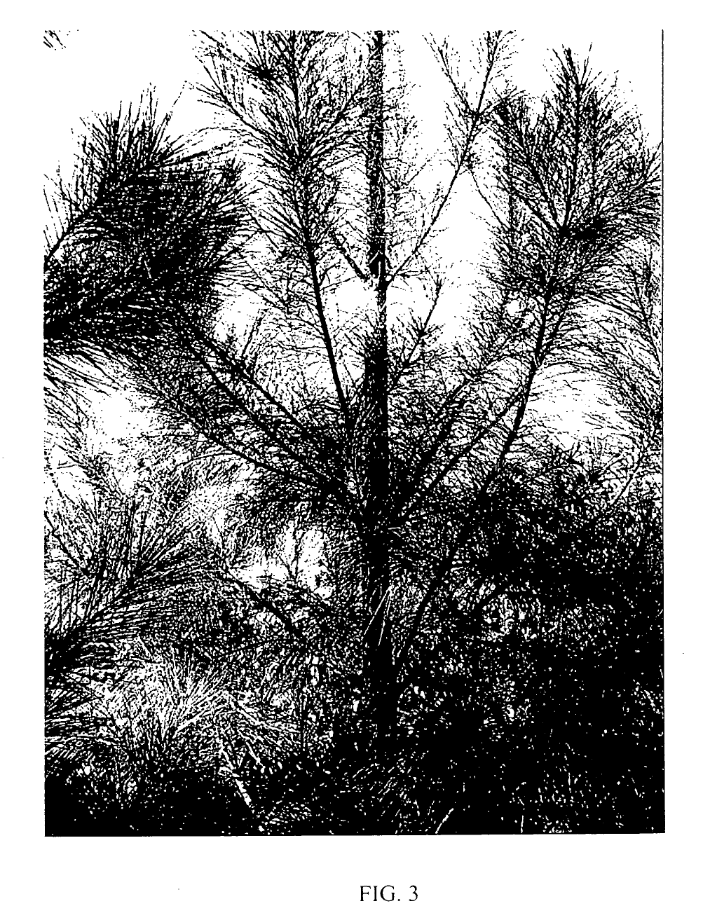

FIG. 4 is a combination of two photographs, with an open-pollinated loblolly pine (checklot 7-56) at left and CF Q3802 at right, showing the straight, single-stemmed nature of CF Q3802, which has a forking rate of 8%, while the open-pollinated loblolly pine tree has a forking rate in excess of 20%.

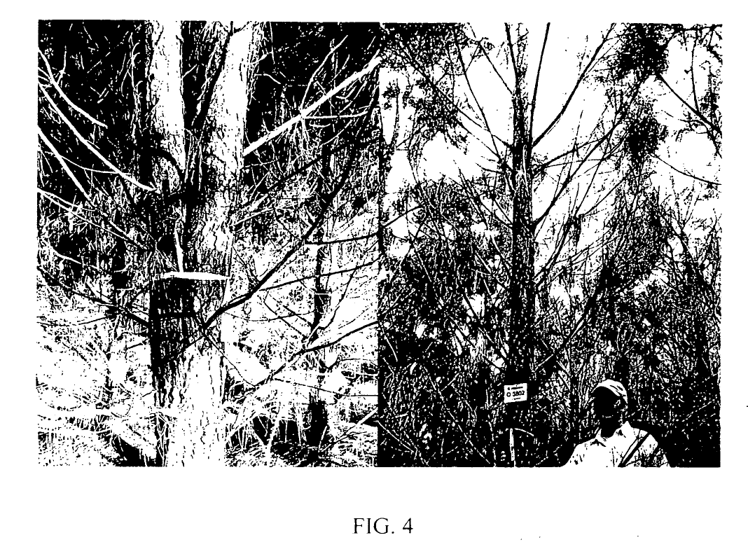

### DETAILED BOTANICAL DESCRIPTION

The botanical details of this new and distinct variety of loblolly pine tree follow:
   Leaf.—Evergreen needles, 6 to 9 inches long, with (usually) three yellow-green needles per fascicle.
   Flower.—Monoecious; males long cylindrical, red to yellow, in clusters at branch tips; females yellow to purple.
   Fruit.—Ovoid to cylindrical, 3 to 6 inch red-brown cones; umbo is armed with a short spine, maturing in early fall.
   Twig.—Orange-brown in color, fine to moderately stout; buds are narrowly ovoid, light reddish brown.
   Bark.—Initially red- to gray-brown and scaly; older trees are ridged and furrowed, with somewhat rounded scaly plates; very old trees have red-brown, flat scaly plates.
   Form.—A medium to large tree can reach well over 100 feet tall, self-prunes well and develops a fairly straight trunk and an oval, somewhat open crown. Compared to unimproved loblolly pine trees, ‘CF Q3802’ is characterized by superior growth, great resistance to fusiform rust (caused by Cronartium quercuum (Berk.) Miyabe ex Shirai f. sp. fusiforme (Cumm.) Burds. et Snow), outstanding stem straightness, long internodes, distinctive narrow crown with slender branches, and low incidence of forking.
   Average height.—27.8 ft after five field growing seasons.
   Maximum height.—36.4 ft after five field growing seasons.
   Average diameter at breast height.—4.7 inches after five field growing seasons.
   Maximum diameter at breast height.—6.0 inches after five field growing seasons.
   Height genetic gain above unimproved trees.—43%.
   Percent stem forking.—8.
   Percent of test trees with ramicorn branches.—0.
   Percent stem fusiform rust field infection.—0.
   Percent branch fusiform rust field infection.—0.
   Percent branch and stem fusiform rust field infection.—0.
   Percent dead ramets due to fusiform rust field infection.—0.
   Percent stem fusiform rust infection in the USDA Resistance Screening Center (Asheville, N.C.) tests after artificial inoculation with rust spores.—2.8 (compared to 75.7% infection in unimproved seedlings).
   Stem form.—very straight.
   Branch angle.—average to steep.
   Branch diameter.—small.
   Number of major whorls.—few.
   Live crown width.—narrow.
   Crown vigor.—average.
   Propagation.—propagated by somatic embryogenesis.
   Use.—high yield industrial plantations.

### Genetic Method of Identification

Microsatellite markers (or SSRs) were used to generate unique DNA profiles (fingerprints) for each genotype produced through somatic embryogenesis, and respective parents. The fingerprint profiles can be used to distinguish between different genotypes. Ten primer pairs (available in public domain; Auckland, L, Bui, T., Zhou, Y., Shepherd, M., and Williams, C. Conifer Microsatellite Handbook; Texas A&M University, College Station, Tex., 2002) were selected as these primer pairs give unique alleles, distinguishable not only from between families, but also within the family of the lines available. Sequences and conditions of SSR primers used in loblolly pine are shown in Table 1.

TABLE 1

Sequences and conditions of SSR primers
currently used in loblolly pine.

| | | | | | |
|---|---|---|---|---|---|
| | | LABEL | | | |
| | | TAIL | | | |
| | | (F/R); | | | |
| PRIMER | | E (end | MgCl₂ | Tm | Size |
| NAME | SEQUENCE (5′-3′) | labeled) | (mM) | (° C.) | (bp) |

| | | | | | |
|---|---|---|---|---|---|
| LOP11 | CCAGAAGGCTATAGTACA | F | 3.0 | 59 | 280 |
| | C; Reverse: CAACAA | | | | |
| | TACAAGTAGCAATAC | | | | |
| PtTx | GCC TTT AGA TGA | R | 5 | 55 | 200 |
| 2037 | ATG AAC CAA; | | | | |
| | Reverse: GGA TAA | | | | |
| | CAA TTT CAC ACA | | | | |
| | GGT AAG CGG GAT | | | | |
| | ATT ATA GAG TTT | | | | |
| PtTx | CAC GAC GTT GTA | F | 2.5 | 59 | 200 |
| 2146 | AAA CGA CCT GGGGAT | | | | |
| | TTG GAT TGG GTA | | | | |
| | TTT G; Reverse: | | | | |
| | ATA TTT TCC TTG | | | | |
| | CCC CTT | | | | |
| | CGA GAC A | | | | |
| PtTx | AAT TTG GGT GTA | R | 2.5 | 54 | 205 |
| 3011 | TTT TTC TTA GA; | | | | |
| | Reverse: GGA TAA | | | | |
| | CAA TTT CAC ACA | | | | |
| | GGA AAA GTT GAA | | | | |
| | GGA GTT GGT GAT C | | | | |
| PtTx | TTC ATC CTA GCT | E | 1.5 | 55 | 471 |
| 3021 | GCT TGC TTT; | | | | |
| | Reverse: CTC AGC | | | | |
| | GTC TAC CCC ATC AA | | | | |
| PtTx | CAC GAC GTT GTA | F | 1.5 | 55 | 225 |
| 3034 | AAA CGA CTC AAA | | | | |
| | ATG CAA AAG ACG; | | | | |
| | Reverse: ATT AGG | | | | |
| | ACT GGG GAT GAT | | | | |
| PtTx | CAC GAC GTT GTA | E | 4 | 59 | 144 |
| 3037 | AAA CGA C; | | | | |
| | Reverse: AAG TCA | | | | |
| | CTT AAT GCA ATA | | | | |
| | TGT A | | | | |
| PtTx | GAA GTG ATA ATG | R | 3 | 55 | 330 |
| 3049 | GCA TAG CAA AAT; | | | | |
| | Reverse: GGA TAA | | | | |
| | CAA TTT CAC ACA | | | | |
| | GGC AGA CCC GTG | | | | |
| | AAA GTA ATA AAC AT | | | | |
| PtTx | TGT CGG TGG AGT | R | 2 | 59 | 280 |
| 3105 | TGG CAG TAG ACT; | | | | |
| | Reverse: GGA TAA | | | | |
| | CAA TTT CAC AC AGG | | | | |
| | GCC CAG CGT TTC | | | | |
| | CTG | | | | |
| PtTx | CAC GAC GTT GTA | F | 2.5 | 59 | 165 |
| 3116 | AAA CGA CCT CCC | | | | |
| | AAA GCC TAA AGA | | | | |
| | AT; Reverse: CAT | | | | |
| | ACA AGG CCT TAT | | | | |
| | CTT ACA GAA | | | | |
| PtTx | TG CAT TCA CCT TGG | E | 3 | 55 | 179 |
| 4054 | AGT T; Reverse: | | | | |
| | TAG GAG ATA ATA | | | | |
| | TAA AAT GTT | | | | |

SSR Analyses

1. DNA was isolated from germinant epicotyl tissue (or young needles in the case of parent material) (˜100 mg) using Qiagen's DNeasy Plant Mini Kit and the Mixer Mill MM 300, as per manufacturer's specifications.

2. DNA was isolated from one line at a time in order to avoid any possible mix-ups or contamination with other lines.

3. DNA was quantified using a spectrophotometer and also quantified visually on agarose gels.

4. DNA was diluted to ca. 10 ng/μl and once again visually inspected.

5. About 50 ng of DNA was used for each PCR reaction. Reactions were carried out in 96 well trays.

6. SSR primer sequences used were as per Texas A & M University (Auckland L., et al., Conifer Microsatellite Handbook, 2002), with the exception that some have a (M13 sequence) tail added. See Table 1.

7. PCR cycles were as follows:

8. 1 cycle at 94° C.=3 minutes

9. 29 cycles at 94° C.=30 seconds; Tm=30 seconds; 72° C.—1 minute.

10. 1 cycle at 72° C.=5 minutes

11. Hold at 4° C.

12. Upon PCR completion, Stop Dye was added, and samples were denatured at 94° C. for 3 minutes.

13. Approximately 0.5-1.0 μl of each sample was loaded on 6.5% polyacrylamide gels and electrophoresed in a LI-COR 4200 DNA Analyzer. Each gel was scored using LI-COR's SAGA software.

14. Selected seedlings were collected in the greenhouse, and each was immediately placed in labeled 1.5 ml locking tube, and placed on dry ice. Seedlings were collected one clonal line at a time to ensure no clonal line mix-ups occurred during sampling. Upon arrival in the lab, tubes were immersed in liquid nitrogen and then frozen at −80° C. until ready for DNA extraction.

Table 2 shows the allele pairs obtained for 35 genotypes, including ‘CF Q3802’, with the various primer pairs used. The fingerprint profile of ‘CF Q3802’ is unique from the other genotypes. Note that primer pairs were used only on those families where the primer pairs yielded informative data. For example, primer pairs PtTx 3037 and PtTx 4054 had a tendency for allele drop out (nulls) and were therefore not included with every family. From this table, one can see that there is little overlap of allele pairs within the various families (distinguished by a different letter prefix), thus each family has a unique fingerprint. The use of ten polymorphic primers allowed us to generate unique fingerprints for each family, for each line, and to confirm their pedigree.

TABLE 2
Alleles obtained per primer set per line.

| | PRIMERS | | | | | | | | | |
|---|---|---|---|---|---|---|---|---|---|---|
| GENOTYPE | PtTx 2037 | | PtTx 2146 | | PtTx 3011-T | | PtTx 3021 | | PtTx 3034 | |
| CF A3643 | 171 | 199 | 189 | 200 | 177 | 233 | 423 | 447 | 224 | 226 |
| CF H3561 | 167 | 167 | 185 | 187 | 201 | 212 | 444 | 489 | 212 | 225 |
| CFJ3599 | 166 | 194 | 191 | 198 | 215 | 230 | 447 | 465 | 219 | 220 |
| CFJ3983 | 166 | 194 | 198 | 209 | 167 | 230 | 447 | 483 | 219 | 220 |
| CF K3357 | 164 | 173 | 192 | 198 | 167 | 248 | 453 | 483 | 219 | 226 |
| CF K3690 | | | 192 | 198 | 176 | 215 | 465 | 483 | 219 | 226 |
| CF K3973 | 164 | 173 | 194 | 198 | 215 | 248 | 465 | 483 | 219 | 226 |
| CF L3243 | | | 189 | 207 | 177 | 227 | 447 | 498 | 224 | 224 |
| CF L3477 | | | 189 | 207 | 177 | 227 | 447 | 498 | 224 | 226 |
| CF L3514 | | | 189 | 198 | 177 | 227 | 423 | 447 | 224 | 224 |
| CF L3519 | | | 189 | 198 | 177 | 227 | 447 | 498 | 224 | 226 |
| CF L3522 | | | 189 | 198 | 177 | 227 | 423 | 432 | 226 | 226 |
| CF L3576 | | | 189 | 198 | 177 | 227 | 447 | 498 | 224 | 224 |
| CF L3642 | | | 189 | 207 | 177 | 227 | 447 | 498 | 224 | 226 |
| CF L3663 | | | 189 | 207 | 177 | 227 | 447 | 498 | 226 | 226 |
| CF L3691 | | | 189 | 198 | 177 | 227 | 447 | 498 | 224 | 224 |
| CF L3771 | | | 189 | 198 | 177 | 227 | 447 | 498 | 224 | 226 |
| CF L3789 | | | 189 | 198 | 177 | 227 | 432 | 498 | 224 | 226 |
| CF L3994 | | | 189 | 198 | 177 | 227 | 447 | 498 | 224 | 226 |
| CF N3407 | 168 | 215 | 187 | 187 | 167 | 209 | 438 | 489 | | |
| CF N3455 | 168 | 168 | 184 | 187 | 209 | 221 | 438 | 462 | | |
| CF N3462 | 168 | 215 | 184 | 187 | 167 | 209 | 438 | 489 | | |
| CF N3773 | 168 | 215 | 184 | 187 | 209 | 221 | 462 | 483 | | |
| CF N3812 | 168 | 215 | 184 | 187 | n/a | n/a | 438 | 489 | | |
| CF N3977 | n/a | n/a | 184 | 187 | 167 | 209 | 438 | 462 | | |
| CF N3984 | 168 | 215 | 187 | 187 | 167 | 167 | 438 | 462 | | |
| CF N3993 | 168 | 215 | 184 | 187 | 167 | 167 | 438 | 462 | | |
| CF O3345 | | | 177 | 198 | 178 | 227 | 432 | 447 | | |
| CF O3621 | | | 177 | 198 | 178 | 227 | 447 | 480 | | |
| CF O3971 | | | 195 | 207 | 184 | 227 | 432 | 480 | | |
| CF Q3472 | 191 | 199 | 182 | 209 | 167 | 233 | 447 | 564 | 224 | 224 |
| CF Q3528 | 191 | 199 | 182 | 209 | 167 | 230 | 477 | 477 | n/a | n/a |
| CF Q3802 | 171 | 191 | 182 | 194 | 167 | 233 | 477 | 486 | 224 | 227 |
| CF Q3880 | 191 | 199 | 194 | 200 | 233 | 233 | 477 | 564 | 224 | 227 |
| CF S3672 | 166 | 167 | | | 209 | 212 | 444 | 462 | | |
| | PRIMERS | | | | | | | | | |
| GENOTYPE | PtTx 3037 | | PtTx 3049 | | PtTx 3105 | | PtTx 3116 | | LOP11 | |
| CF A3643 | | | 318 | 320 | 193 | 199 | 148 | 149 | | |
| CF H3561 | 134 | 164 | 320 | 320 | 190 | 193 | 152 | 158 | | |
| CFJ3599 | | | 320 | 330 | 202 | 220 | 149 | 155 | | |
| CFJ3983 | | | 320 | 330 | 193 | 202 | 149 | 155 | | |
| CF K3357 | n/a | n/a | 328 | 332 | 196 | 220 | 150 | 183 | | |
| CF K3690 | 174 | 210 | 326 | 328 | 193 | 220 | 147 | 150 | | |
| CF K3973 | 142 | 142 | 326 | 332 | 193 | 193 | 150 | 183 | | |
| CF L3243 | | | 322 | 328 | 193 | 205 | 150 | 162 | | |
| CF L3477 | | | 316 | 324 | 193 | 205 | 150 | 162 | | |
| CF L3514 | | | 316 | 324 | 193 | 205 | 150 | 156 | | |
| CF L3519 | | | 322 | 328 | 193 | 202 | 150 | 162 | | |
| CF L3522 | | | 316 | 324 | 193 | 202 | 147 | 162 | | |
| CF L3576 | | | 322 | 328 | 193 | 202 | 147 | 162 | | |
| CF L3642 | | | 322 | 328 | 193 | 205 | 147 | 156 | | |
| CF L3663 | | | 316 | 324 | 193 | 205 | 150 | 162 | | |
| CF L3691 | | | 316 | 324 | 193 | 202 | 147 | 156 | | |
| CF L3771 | | | 316 | 324 | 193 | 202 | 147 | 162 | | |
| CF L3789 | | | 322 | 328 | 193 | 202 | 150 | 156 | | |
| CF L3994 | | | N/A | N/A | 193 | 202 | 150 | 162 | | |
| CF N3407 | 113 | 137 | 326 | 328 | 193 | 214 | 147 | 164 | | |
| CF N3455 | 113 | 137 | 328 | 330 | 193 | 202 | 147 | 161 | | |
| CF N3462 | 113 | 137 | 322 | 326 | 193 | 202 | 147 | 161 | | |
| CF N3773 | 131 | 140 | 316 | 328 | 193 | 202 | 147 | 152 | | |
| CF N3812 | 113 | 137 | 328 | 330 | 193 | 202 | 147 | 164 | | |
| CF N3977 | 131 | 164 | 322 | 330 | 193 | 214 | 147 | 161 | | |
| CF N3984 | 131 | 164 | 326 | 328 | n/a | n/a | 147 | 164 | | |
| CF N3993 | 113 | 137 | 328 | 330 | 193 | 202 | 147 | 161 | | |
| CF O3345 | | | | | 202 | 205 | 149 | 157 | | |
| CF O3621 | | | | | 202 | 202 | 149 | 157 | | |
| CF O3971 | | | | | 205 | 217 | 146 | 157 | | |
| CF Q3472 | 114 | 150 | 318 | 320 | 196 | 202 | 151 | 151 | 271 | 279 |
| CF Q3528 | 192 | 192 | 318 | 322 | 193 | 193 | 151 | 160 | 271 | 279 |
| CF Q3802 | 150 | 192 | 318 | 322 | 193 | 193 | 151 | 160 | 271 | 279 |
| CF Q3880 | 150 | 192 | 318 | 322 | 193 | 202 | 151 | 160 | 271 | 289 |
| CF S3672 | 147 | 156 | 320 | 326 | 253 | 253 | 152 | 158 | | |

Although the new variety of loblolly pine tree possesses the detailed characteristics noted above as a result of the growing conditions prevailing in the seven test locations, it is to be understood that the variations of the usual magnitude and characteristics incident to changes in growing conditions, irrigation, fertilization, pruning, pest control, climatic variations and the like are to be expected. An example of ‘CF Q3802’ can be found at Plum Creek Glynn Farms year 2000 line trial, Jessup, Ga.

Having thus described and illustrated our new variety of loblolly pine tree, what we claim: as new and desire to secure by Plant Letter Patent is:

1. A new and distinct variety of loblolly pine tree named CF Q3802 substantially as described and illustrated.
